(19) United States
(12) Patent Application Publication
(10) Pub. No.: US 2012/0115774 A1
(43) Pub. Date: May 10, 2012

(54) ANTIMICROBIAL CATIONIC LIPO-BETA-PEPTIDES

(75) Inventors: George Zhanel, Winnipeg (CA); Frank Schweizer, Winnipeg (CA)

(73) Assignee: UNIVERSITY OF MANITOBA, Winnipeg, MB (CA)

(21) Appl. No.: 13/260,670

(22) PCT Filed: Feb. 23, 2010
(86) PCT No.: PCT/IB2010/000475

§ 371 Date: Dec. 19, 2011

Related U.S. Application Data
(60) Provisional application No. 61/164,117, filed on Mar. 27, 2009.

(51) Int. Cl. A61K 38/07; A61P 31/04; C07K 5/11

(52) U.S. Cl. 514/2.6; 530/330; 514/3.2; 514/2.7; 514/2.8

(57) ABSTRACT

In some aspects, the present invention provides ultrashort lipopeptides which feature β-amino acids or β-peptides and demonstrate antimicrobial activity. Accordingly, some aspects of the present invention provide lipopeptide compositions and methods of making and using the compositions as antimicrobial agents.

This application claims priority to U.S. Application No. 61/164,117 filed on Mar. 27, 2009, the entire disclosure of which is specifically incorporated herein by reference in its entirety without disclaimer.

### BACKGROUND OF THE INVENTION

I. Field of the Invention

The present invention relates generally to antimicrobial drug development. More particularly, it concerns improvements in the therapeutic potential of cationic lipopeptides as antimicrobial agents.

II. Description of Related Art

The rise of antibiotic resistant microbes has prompted interest in novel therapeutics with new modes of action, including antimicrobial lipopeptides. Naturally occurring lipopeptides produced by bacteria, yeast and fungi exist with largely antifungal activity, but some also show antibacterial activity (Avrahami and Shai, 2003; Jerala, 2007; Makovitzki et al., 2006). Native lipopeptides are cyclic, anionic and contain short peptide portions of 6-7 D- and L-amino acids that are toxic to mammalian cells due to a lack of selectivity (Avrahami and Shai, 2004; Shai et al., 2006). However, studies of synthetic lipopeptides formed from acylated antimicrobial peptides report a marked improvement in bioactivity against bacteria (Andra et al., 2005; Japelj et al., 2007; Majerle et al., 2003).

Recently, a series of short lipopeptides were synthesized from biologically inactive D,L cationic tetrapeptides and were found to possess cell-lysing activity against a variety of Gram positive and Gram negative bacteria, with both aliphatic chain length and peptide sequence determining the cell type selectivity (Makovitzki et al., 2006). However, given the continued rise of antibiotic resistant microbes, there remains a need for novel lipopeptide antibiotics.

### SUMMARY OF THE INVENTION

In some aspects, the present invention stems in part form the recent discovery by the inventors of pharmacokinetically improved ultrashort lipopeptides which feature beta-amino acids (β-amino acids or β-peptides) and demonstrate antimicrobial activity. Accordingly, some aspects of the present invention provide lipopeptide compositions and methods of making and using the compositions as antimicrobial agents.

In one aspect, the present invention provides a cationic lipo-β-ultrashort peptide of formula (I):

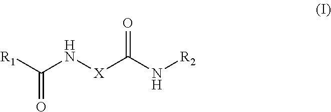

where R₁ is a hydrophobic group; R₂ is H, alkyl group or amide protecting group and —NH—X—C(O)— is a chain of two to four β-amino acid residues; or a salt thereof. In some embodiments, R₁ is alkyl_{(C5-45)} or alkenyl_{(C5-45)}. In some embodiments, R₁ is alkyl_{(C10-18)}. In some embodiments, R₁ is alkyl_{(C12-16)}. In some embodiments, R₂ is H. In some embodiments, the cationic lipo-β-ultrashort peptide is present as a trifluoroacetic acid salt. In some embodiments, the ultrashort β-peptide comprises one or more cationic β-amino acids. In particular embodiments, each of the two to four amino acid residues of the —NH—X—C(O)— group is selected from the group consisting of β-lysine, β-arginine, β-alanine, β-glycine, β-leucine and β-histidine.

In some embodiments, the cationic lipo-β-ultrashort peptide is a tetrapeptide. In some embodiments, —NH—X—C(O)— is a chain of four β-amino acid residues. In some of these embodiments, the chain of four β-amino acid residues is KGGK (SEQ ID NO:1), KKKK (SEQ ID NO:2), KAAK (SEQ ID NO:3), or KLLK (SEQ ID NO:4). In further embodiments, the chain of four β-amino acid residues is (β) C16-KGGK, (β) C16-KKKK, (β) C16-KAAK, or (β) C12-KLLK. In some embodiments, the cationic lipo-β-ultrashort peptide is a compound as shown in FIG. 2A, 2B, 2C, or 2D.

In another aspect, the present invention provides methods of making a cationic Lipo-β-ultrashort peptide. In some embodiments, the present invention provides method of making a cationic Lipo-β-ultrashort peptide of formula (I):

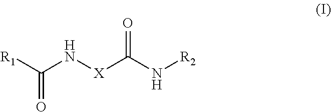

where: R₁ is a hydrophobic group; R₂ is H, alkyl group or amide protecting group and —NH—X—C(O)— is a cationic ultrashort β-peptide; or a salt thereof comprising reacting a fatty acid of formula (II):

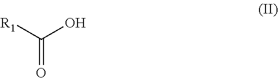

where: R₁ is a hydrophobic group; with the N-terminus of a cationic ultrashort β-peptide of formula (III):

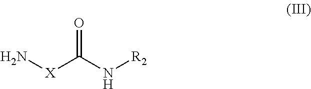

where: R₂ is H, alkyl group or an amide protecting group, and —NH—X—C(O)— is a cationic ultrashort β-peptide; or a salt thereof, under conditions to form a cationic lipo-β-ultrashort peptide of formula (I). In particular embodiments, the conditions comprise a solid-phase synthesic step. In some embodiments, the number of equivalents of fatty acid to cationic ultrashort β-peptide ranges from about 1-5. In some embodiments, the method further comprises obtaining the cationic ultrashort β-peptide. In other embodiments, the method may comprise preparing the cationic ultrashort β-peptide via solid-phase synthesis. In some embodiments, the cationic lipo-β-ultrashort peptide is present as a trifluoroacetic acid salt.

In certain embodiments, the present invention encompasses pharmaceutical composition comprising a cationic lipo-β-ultrashort peptide of formula (I):

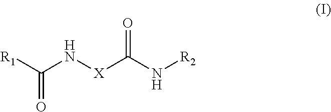

where: R₁ is a hydrophobic group; R₂ is H, alkyl or an amide protecting group, and —NH—X—C(O)— is a chain of two to four β-amino acid residues; or a salt thereof; and a pharmaceutically acceptable carrier. The pharmaceutical composition disclosed herein may be formulated to be administered in any appropriate formulation. In certain embodiments, the pharmaceutical composition is formulated to be administered topically.

In a further aspect, the present invention provides a method of treating a bacterial infection in a subject comprising administering to the subject an effective amount of a cationic lipo-β-ultrashort peptide as disclosed herein. In some embodiments, the method may further comprise diagnosing the subject as needing treatment for the bacterial infection prior to administering the cationic lipo-β-ultrashort peptide. In some embodiments, the method may further comprise administration of a second antibacterial agent.

In some embodiments, the effective amount is ≦1, 2, 4, 8, 16, 32, 64, 128, or 256 μg/mL, or any range derivable therebetween. In certain embodiments, the effective amount (MIC) is ≦64 μg/mL. In some embodiments, the cationic lipo-β-ultrashort peptide of formula (I) may be employed in a method of the present invention such that the effective amount of the cationic lipo-β-ultrashort peptide is less than the effective amount of the corresponding cationic lipo-α-ultrashort peptide.

In some embodiments, the bacterial infection is an infection by a Gram-positive bacteria or a Gram-negative bacteria. In select embodiments, the bacterial infection is an infection by a Gram-positive bacteria selected from the group consisting of Staphylococcus aureus, methicillin-resistant Staphylococcus aureus (MRSA), Staphylococcus epidermidis, methicillin-resistant S. epidermidis (MRSE), Enterococcus faecalis, Enterococcus faecium, and Streptococcus pneumoniae. In certain embodiments, the Gram-positive bacteria is Staphylococcus epidermidis or methicillin-resistant Staphylococcus epidermidis (MRSE). In other embodiments, the bacterial infection is may be an infection by a Gram-negative bacteria selected from the group consisting of E. coli, Pseudomonas aeruginosa, Stenotrophomonas maltophilia, Acinetobacter baumannii, and Klebsiella pneumoniae. In certain embodiments, the Gram-negative bacteria is E. coli. In some embodiments, the bacteria causing the bacterial infection is a multi-drug resistant bacteria.

The pharmaceutical composition may be administered in any appropriate manner known to those of skill in the art. In some embodiments, the pharmaceutical composition may be administered topically, intravenously, intradermally, intraarterially, intraperitoneally, intralesionally, intracranially, intraarticularly, intraprostaticaly, intrapleurally, intratracheally, intravitreally, intravaginally, intrarectally, intratumorally, intramuscularly, intraperitoneally, subcutaneously, subconjunctivally, intravesicularly, mucosally, intrapericardially, intraumbilically, intraocularally, intrathecally, locally, by injection, by infusion, by localized perfusion bathing target cells directly, via a catheter, or via a lavage. In particular embodiments, the pharmaceutical composition is administered topically to skin of the subject.

In another aspect, the present invention provides a method of preventing a bacterial infection in a subject comprising administering to the subject an effective amount of a cationic lipo-β-ultrashort peptide as disclosed herein. In some embodiments, the method may further comprise diagnosing the subject as needing preventative treatment for the bacterial infection prior to administering the cationic lipo-β-ultrashort peptide.

Embodiments discussed in the context of methods and/or compositions of the invention may be employed with respect to any other method or composition described herein. Thus, an embodiment pertaining to one method or composition may be applied to other methods and compositions of the invention as well.

When used in the context of a chemical group, “hydrogen” means —H; “hydroxy” means —OH; “oxo” means ═O; “halo” means independently —F, —Cl, —Br or —I; “amino” means —NH₂; “hydroxyamino” means —NHOH; “nitro” means —NO₂; imino means ═NH; “cyano” means —CN; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂—; “sulfonyl” means —S(O)₂— (see below for definitions of groups containing the term sulfonyl, e.g., alkylsulfonyl); “sulfinyl” means —S(O)—; and “silyl” means —SiH₃.

For the groups below, the following parenthetical subscripts further define the groups as follows: “(Cn)” defines the exact number (n) of carbon atoms in the group. “(C≦n)” defines the maximum number (n) of carbon atoms that can be in the group, with the minimum number of carbon atoms in such at least one, but otherwise as small as possible for the group in question, e.g., it is understood that the minimum number of carbon atoms in the group “alkenyl_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn−n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

A hydrophobic group is a chemical group that is significantly non-polar and exhibits a tendency to dissolve in nonpolar solvents, such as hexane or toluene

As used herein, “lipid” or “lipo” refers to a straight-chain hydrocarbon radical having 5 carbons or higher, wherein the radical may comprise single, double, and/or triple bonds.

As used herein, “ultrashort” peptides refer to di-, tri- and tetra-peptides.

As used herein, “β-peptide” refers to peptides that differ from α-peptides by having a methylene (CH₂) group inserted into every amino acid residue, either between the C═O group and the α-carbon atom (β³) or between the α-carbon and the nitrogen atom (β²).

“Peptide” refers to two or more amino acids joined together by an amide bond. As used herein, the term “amino acid” refers both to the naturally occurring amino acids and their derivatives. In addition, a mimic of one or more amino acids, otherwise known as peptide mimetic or peptidomimetic can also be used. In some aspects, the invention further contemplates that the N-terminus and/or C-terminus may be modified with respect to a native peptide, or it may be unmodified. Generally, the modification include, but are not limited to, activating, deactivating, protecting, or deprotecting one or both terminal ends. The invention also contemplates modifications that make the terminus more or less reactive.

“Cationic lipo-β-ultrashort peptide” incorporates the above definitions, wherein at least two of the β-amino acids each have at least one positive charge.

As used herein, “protecting group” refers to a moiety attached to a functional group to prevent an otherwise unwanted reaction of that functional group. The term “functional group” generally refers to how persons of skill in the art classify chemically reactive groups. Examples of functional groups include hydroxyl, amine, sulfhydryl, amide, carboxyl, carbonyl, etc. Protecting groups are well-known to those of skill in the art. Non-limiting exemplary protecting groups fall into categories such as hydroxy protecting groups, amino protecting groups, sulfhydryl protecting groups and carbonyl protecting groups. Such protecting groups, including examples of their installation and removal, may be found in Greene and Wuts, 1999, incorporated herein by reference in its entirety. Triazole aminoglycoside-(amino acid)ₙ conjugates described herein are contemplated as protected by one or more protecting groups—that is, the present invention contemplates such conjugates in their “protected form.” Non-limiting examples of carboxylic acid protecting groups include benzyl (Bn) and t-butyl. Non-limiting examples of amino protecting groups include Bn, carbobenzyloxy (Cbz), t-butoxycarbonyl (Boc) and 9-fluorenylmethyloxycarbonyl (Fmoc), for example.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults, juveniles, infants and fetuses.

As used herein, a “multidrug resistant (MDR) bacteria” is resistant to two or more antimicrobial classes.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-H: Exemplary cationic lipo-α-peptides. The position of the D-enantiomers is shown by bold and underlining. 1A: (α) C16-KGGK C₃₈H₆₃F₉N₇O₈ Exact Mass: 916.4594 Mol. Wt.: 916.9345 Final product (mass): 32 mg. 1B: (α) C16-KGGK C₃₈H₆₃F₉N₇O₈ Exact Mass: 916.4594 Mol. Wt.: 916.9345 Final product (mass): 84 mg. 1C: (α) C16-KKKK C₅₀H₈₁F₁₅N₉O₁₀ Exact Mass: 1252.5867 Mol. Wt.: 1253.2085 Final product (mass): 45 mg. 1D: (a) C16-KKKK C₅₀H₈₁F₁₅N₉O₁₀ Exact Mass: 1252.5867 Mol. Wt.: 1253.2085 Final product (mass): 87 mg. 1E: (α) C16-KAAK C₄₀H₆₇F₉N₇O₈ Exact Mass: 944.4907 Mol. Wt.: 944.9877 Final product (mass): 34 mg. 1F: (α) C16-KAAK C₄₀H₆₇F₉N₇O₈ Exact Mass: 944.4907 Mol. Wt.: 944.9877 Final product (mass): 40 mg. 1G: (α) C12-KLLK C₄₂H₇₁F₉N₇O₈ Exact Mass: 972.5220 Mol. Wt.: 973.0409 Final product (mass): 123 mg. 1H: (α) C12-KLLK C₄₂H₇₁F₉N₇O₈ Exact Mass: 972.5220 Mol. Wt.: 973.0409 Final product (mass): 105 mg.

FIGS. 2A-D: Exemplary cationic lipo-β-peptides. 2A: (β) C16-KGGK C₄₂H₇₁F₉N₇O₈ Exact Mass: 972.5220 Mol. Wt.: 973.0409 Final product (mass): 75 mg. 2B: (β) C16-KKKK C₅₄H₈₉F₁₅N₉O₁₀ Exact Mass: 1308.6493 Mol. Wt.: 1309.3148 Final product (mass): 88 mg. 2C: (β) C16-KAAK C₄₄H₇₅F₉N₇O₈ Exact Mass: 1000.5533 Mol. Wt.: 1001.0940 Final product (mass): 72 mg. 2D: (β) C12-KLLK C₄₆H₇₉F₉N₇O₈ Exact Mass: 1028.5846 Mol. Wt.: 1029.1472 Final product (mass): 91 mg

### DESCRIPTION OF THE ILLUSTRATIVE EMBODIMENTS

Recently, a series of short lipopeptides were synthesized from biologically inactive D,L cationic tetrapeptides and were found to possess cell-lysing activity against a variety of Gram positive and Gram negative bacteria, with both aliphatic chain length and peptide sequence determining the cell type selectivity (Makovitzki et al., 2006). Here, the biological activity of short cationic lipo-β-peptides is demonstrated. As with the incorporation of D-enantiomers, peptidomimetics incorporating β-amino acids offers the potential benefit of metabolic and enzymatic stability against proteases, one of the major drawbacks in peptide-based drug development (Steer et al., 2002).

As resistance to lipopeptides is a generally rare occurrence, and with the advantages β-amino acids provide, lipo-β-peptides provide novel therapeutics (Straus and Hancock, 2006; Steer et al., 2002; Seebach et al., 2004). These results demonstrate that lipo-β-peptides display antimicrobial activity comparable to that of lipo-α-peptides. Previous studies have shown that the mode of action of ultrashort α-lipopeptides involves permeation and disintegration of membranes, similar to that of many long antimicrobial peptides (Makovitzki et al., 2006). This mode of action makes it difficult for the microorganisms to develop resistance. It is unlikely that these ultrashort α- and β-lipopeptides will form a defined and stable amphipathic structure. This implies that ultrashort α- and β-lipopeptides will retain similar mode of antibacterial action.

### A. LIPOPEPTIDES

1. General

Generally speaking, a lipopeptide is a molecule consisting of a lipid connected to a peptide. Native lipopeptides are cyclic, anionic and contain short peptide portions of 6-7 D- and L-amino acids that are toxic to mammalian cells due to a lack of selectivity (Avrahami and Shai, 2004; Shai, et al., 2006). However, studies of synthetic lipopeptides formed from acylated antimicrobial peptides report a marked improvement in bioactivity against bacteria (Andra, et al., 2005; Japelj et al., 2007; Majerle et al., 2003).

Some lipopeptides are used as antibiotics. Lipopeptide antibiotics represent an old class of antibiotics and generally consist of a hydrophilic cyclic peptide portion attached to a fatty acid chain.

a. Lipids

In some embodiments of the present invention, the hydrophobic group may be a lipid. As used herein, “lipid” or “lipo” refers to a straight-chain hydrocarbon radical having 5 carbons or higher, wherein the radical may comprise single, double, and/or triple bonds. Examples include compounds which contain long-chain aliphatic hydrocarbons and their derivatives. In some embodiments, R₁ is alkyl_{(C5-45)} or alkenyl_{(C5-45)}. In some embodiments, R₁ is alkyl_{(C10-18)}. In some embodiments, R₁ is alkyl_{(C12-16)}. In a particular embodiment, the hydrophobic group is the fatty acid palmitic acid. In other embodiments, the hydrophobic group is the fatty acid lauric acid.

b. Peptides

“Peptide” refers to two or more amino acids joined together by an amide bond. As used herein, the term “amino acid” refers both to the naturally occurring amino acids and their derivatives. These peptides include the twenty “natural” amino acids, and post-translational modifications thereof.

Additionally, structurally similar compounds may be formulated to mimic the key portions of peptide or polypeptides of the present invention. Such compounds, which are termed peptidomimetics, may be used in the same manner as the peptides of the invention. As used herein, the term “mimic” means an amino acid or an amino acid analog that has the same or similar functional characteristic of an amino acid. A peptide mimetic or peptidomimetic is an organic molecule that retains similar peptide chain pharmacophore groups as are present in the corresponding peptide. The substitution of amino acids by non-naturally occurring amino acids or peptidomimetics as described above can enhance the overall activity or other properties of an individual peptide based on the modifications of the side chain functionalities. For example, these types of modifications to the exemplified peptides can enhance the peptide's stability to enzymatic breakdown or increase biological activity. A person having ordinary skill in the art would be familiar with these functional equivalents.

c. β-Peptides

β-peptides consist of 0 amino acids, which have their amino group bonded to the 0 carbon rather than the α carbon as in the 20 standard biological amino acids. The only commonly naturally occurring β amino acid is β-alanine Although it is used as a component of larger bioactive molecules, β-peptides in general do not appear in nature.

In α amino acids, both the carboxylic acid group and the amino group are bonded to the same carbon center, termed the α carbon because it is one atom away from the carboxylate group. In β amino acids, the amino group is bonded to the β carbon, which is found in most of the 20 standard amino acids. Only glycine lacks a β carbon, which means that β-glycine is not possible. Two main types of β-peptides exist: those with the organic residue (R) next to the amine are called β²-peptides and those with position next to the carbonyl group are called β²-peptides. β-peptides are stable against proteolytic degradation in vitro and in vivo, an important advantage over natural peptides in the preparation of peptide-based drugs. In particular embodiments, the β-peptide comprises one or more cationic β-amino acids which may be selected from the group consisting of β-lysine, β-alanine, β-glycine, β-leucine and β-histidine.

2. Lipoprotein Synthesis

In another aspect, the present invention provides methods of making a cationic lipo-β-ultrashort peptide. In some embodiments, the present invention provides method of making a cationic lipo-β-ultrashort peptide of formula (I):

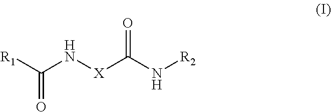

where: R₁ is a hydrophobic group; R₂ is H, alkyl or amide protecting group, and —NH—X—C(O)— is a cationic ultrashort β-peptide; or a salt thereof comprising reacting a fatty acid of formula (II):

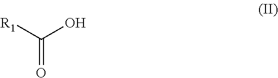

where: R₁ is a hydrophobic group; with the N-terminus of a cationic ultrashort β-peptide of formula (III):

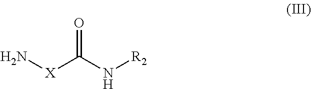

where: R₂ is H, alkyl or an amide protecting group, and —NH—X—C(O)— is a cationic ultrashort β-peptide; or a salt thereof, under conditions to form a cationic lipo-β-ultrashort peptide of formula (I). One skilled in the art can easily synthesize the peptides and lipopeptides of this invention. Standard procedures for preparing synthetic peptides are well known in the art. See, e.g., U.S. Pat. No. 6,911,525. For example, peptides of the invention can be synthesized by such commonly used methods as t-BOC or FMOC protection of alpha-amino groups. Both methods involve stepwise syntheses whereby a single amino acid is added at each step starting from the carboxyl-terminus of the peptide (See, Coligan et al., 1991).

In particular embodiments, the conditions comprise a solid-phase synthesic step. Peptides of the invention can be synthesized by the solid phase peptide synthesis methods well known in the art. (Merrifield, 1963), and Stewart and Young, 1984). For example, peptides can be synthesized using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about 0.25 to 1 hour at 0° C. After evaporation of the reagents, the peptides are extracted from the polymer with 1% acetic acid solution which is then lyophilized to yield the crude material. This can typically be purified by techniques such as gel filtration on Sephadex G-15 using 5% acetic acid as a solvent, by high pressure liquid chromatography, and the like. Lyophilization of appropriate fractions of the column will yield the homogeneous peptide or peptide derivatives, which can then be characterized by standard techniques such as amino acid analysis, thin layer chromatography, high performance liquid chromatography, ultraviolet absorption spectroscopy, molar rotation, solubility, and assessed by the solid phase Edman degradation (See e.g. Deutscher, 1990). Automated synthesis using FMOC solid phase synthetic methods can be achieved using an automated peptide synthesizer (Model 432A, Applied Biosystems, Inc.).

In other aspects of the invention, the peptides of the present invention can also be synthesized using a fusion protein microbial method in which an anionic carrier peptide is fused to a cationic peptide. A method for such microbial production of cationic peptides having anti-microbial activity is provided in U.S. Pat. No. 5,593,866. The peptide of the present invention thus produced can be purified by isolation/purification methods for proteins generally known in the field of protein chemistry. More particularly, there can be mentioned, for example, extraction, recrystallization, salting out with ammonium sulfate, sodium sulfate, etc., centrifugation, dialysis, ultrafiltration, adsorption chromatography, ion exchange chromatography, hydrophobic chromatography, normal phase chromatography, reversed-phase chromatography, gel filtration method, gel permeation chromatography, affinity chromatography, electrophoresis, countercurrent distribution, etc. and combinations of these. Most effective is a method by reversed-phase high performance liquid chromatography.

The peptide of the present invention may form a salt by addition of an acid. Examples of the acid include inorganic acids (such as trifluoroacetic acid, hydrochloric acid, hydrobromic acid, phosphoric acid, nitric acid, and sulfuric acid) or organic carboxylic acids (such as acetic acid, propionic acid, maleic acid, succinic acid, malic acid, citric acid, tartaric acid, and salicylic acid), acidic sugars such as glucuronic acid, galacturonic acid, gluconic acid, ascorbic acid, etc., acidic polysaccharides such as hyaluronic acid, chondroitin sulfates, alginic acid, or organic sulfonic acids (such as methanesulfonic acid, and p-toluenesulfonic acid), and the like. In some embodiments, the salt is a pharmaceutically acceptable salt. In particular embodiments, the cationic lipo-β-ultrashort peptide is present as a trifluoroacetic acid salt.

In some embodiments, the peptide of the present invention may form a salt with a basic substance. Examples of the salt include, for example, pharmaceutically acceptable salts selected from salts with inorganic bases such as alkali metal salts (sodium salt, lithium salt, potassium salt etc.), alkaline earth metal salts, ammonium salts, and the like or salts with organic bases, such as diethanolamine salts, cyclohexylamine salts and the like.

### B. BACTERIAL INFECTIONS

In certain aspects of the invention, the compositions are useful for the treatment and/or prevention of bacterial infections. The bacterial infection may be caused by a gram positive or gram negative bacterium. In some embodiments, the bacteria causing the bacterial infection is a multi-drug resistant bacteria.

The term “gram-negative bacteria” or “gram-negative bacterium” as used herein is defined as bacteria which have been classified by the Gram stain as having a red stain. Gram-negative bacteria have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary Gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “gram-positive bacteria” or “gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

### C. PHARMACEUTICAL COMPOSITIONS

In some aspects, the present invention provides a pharmaceutical composition comprising a cationic lipopeptide and a pharmaceutically acceptable carrier. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, therapeutic compositions is contemplated. Supplementary active ingredients, such as other anti-microbial agents, can also be incorporated into the compositions.

The pharmaceutical compositions comprising the cationic lipo-β-peptide may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (See, for example, Remington's Pharmaceutical Sciences, 18th Ed. Mack Printing Company, 1990, incorporated herein by reference). In particular embodiments, the pharmaceutical composition is formulated to be and is administered topically.

The pharmaceutical pharmaceutical compositions comprising the cationic lipopeptide may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for topical administrations or formulated for alimentary administrations such as drug release capsules and the like.

Further in accordance with some aspects of the present invention, the composition suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art. In one embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### D. THERAPEUTIC APPLICATIONS

In certain aspects of the invention, the compositions are useful for the treatment and/or prevention of bacterial infections. In some embodiments, the present invention provides a method of treating and/or preventing a bacterial infection comprising administering to the subject an effective amount of a cationic lipopeptide.

The term “effective concentration” or “effective amount” means that a sufficient amount of the antimicrobial agent is added to decrease, prevent or inhibit the growth of bacterial organisms or bacterial colonization. The term “inhibiting” or “reducing” as used herein, is taken to mean the act of limiting the growth of microbes or pathogenic bacteria. The amount will vary for each compound and upon known factors such as pharmaceutical characteristics; the type of medical device; age, sex, health and weight of the recipient; and the use and length of use. It is within the skilled artisan's ability to relatively easily determine an effective concentration for each compound. In some embodiments, the cationic lipo-β-ultrashort peptide may be employed in a method of the present invention such that the effective amount of the cationic lipo-β-ultrashort peptide is less than the effective amount of the corresponding cationic lipo-α-ultrashort peptide. In some embodiments, the effective amount is between 1 and 256 μg/mL, or any range derivable in between. In particular embodiments, the effective amount is <64 μg/mL. In other embodiments, the effective amount is 1, 2, 4, 8, 16, 32, 64, 128, or 256 μg/mL.

The terms “treat,” “treating” or “treatment,” as used herein, include alleviating, abating or ameliorating a bacterial infection, symptoms, preventing additional symptoms, inhibiting the infection, e.g., arresting the development of the infection, relieving the infection, causing regression of the infection, relieving a condition caused by the infection, or stopping the symptoms of the infection either prophylactically and/or therapeutically.

“Prevention” or “preventing” includes: (1) inhibiting the onset of a disease, condition, or infection in a subject or patient which may be at risk and/or predisposed to the disease, condition, or infection but does not yet experience or display any or all of the pathology or symptomatology of the disease, condition, or infection, and/or (2) slowing the onset of the pathology or symptomatology of a disease, condition, or infection in a subject of patient which may be at risk and/or predisposed to the disease, condition, or infection but does not yet experience or display any or all of the pathology or symptomatology of the disease, condition, or infection.

In further embodiments, the method further comprises diagnosing the subject as needing treatment for the bacterial infection prior to administering the composition. Such diagnostic methods are well known to those having skill in the art.

1. Treatment Regimens

Treatment regimens may vary as well, and depend on the stage of bacterial infection and its consequences. The clinician will be best suited to make decisions on the best regimen to use based on the positive determination of the existing bacterial infection, the use of antibiotics and the known efficacy and toxicity (if any) of the therapeutic formulations.

The improvement is any observable or measurable improvement. Thus, one of skill in the art realizes that a treatment may improve the patient or subject's condition, but may not be a complete cure of the disease.

The composition of the present invention is utilized to markedly inhibit, reduce, prevent, abrogate, or minimize bacterial colonization, bacterial translocaton and/or bacterial invasion into host tissues. Reduction, abrogation, minimization or prevention of microbial growth is achieved by using an effective concentration such that the concentration is effective to reduce the growth or colonization or translocation into host tissues or invasion into host of the microbes by at least 10%, 20%, 30%, 40%, 50%, 60%, 70%, 80%, 90%, 100% or any range therebetween.

2. Combination Treatments

In order to increase the effectiveness of the composition, it may be desirable to combine these compositions and methods of the invention with a known agent effective in the treatment or prevention of bacterial infections, e.g., antibiotics. Antibacterial agents and classes thereof that may be co-administered with a compound of the present invention include, without limitation, penicillins and related drugs, carbapenems, cephalosporins and related drugs, aminoglycosides, bacitracin, gramicidin, mupirocin, chloramphenicol, thiamphenicol, fusidate sodium, lincomycin, clindamycin, macrolides, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, eveminomicin, glycopeptide, glycylcycline, ketolides, oxazolidinone; imipenen, amikacin, netilmicin, fosfomycin, gentamicin, ceftriaxone, Ziracin, LY 333328, CL 331002, Linezolid, Synercid, Aztreonam, Metronidazole, Epiroprim, OCA-983, GV-143253, Sanfetrinem sodium, CS-834, Biapenem, A-99058.1, A-165600, A-179796, KA 159, Dynemicin A, DX8739, DU 6681; Cefluprenam, ER 35786, Cefoselis, Sanfetrinem celexetil, HGP-31, Cefpirome, HMR-3647, RU-59863, Mersacidin, KP 736, Rifalazil; Kosan, AM 1732, MEN 10700, Lenapenem, BO 2502A, NE-1530, PR 39, (L-arginyl-L-ariginyl-L-arginyl-L-prolyl-L-arginyl-L-prolyl-L-prolyl-L-try osyl-L-leucyl-L-prolyl-L-arginyl-L-prolyl-L-arginyl-L-prolyl-L-prolyl-L-pro lyl-L-phenylalanyl-L-phenylalanyl-L-prolyl-L-prolyl-L-arginyl-L-leucyl-L-pr olyl-L-prolyl-L-arginyl-L-isoleucyl-L-prolyl-L-prolylglycyl-L-phenylalanyl-L-prolyl-L-prolyl-L-arginyl-L-phenyalanyl-L-prolyl-L-prolyl-L-arginyl-L-phenylalanyl-L-prolinamide), K130, OPC 20000, OPC 2045, Veneprim, PD 138312, PD 140248, CP 111905, Sulopenem, ritipenam acoxyl, RO-65-5788, Cyclothialidine, Sch-40832, SEP-132613, micacocidin A, SB-275833, SR-15402, SUN A0026, TOC 39, carumonam, Cefozopran, Cefetamct pivoxil, and T 3811.

In some embodiments, the composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination. In other aspects, one or more agents may be administered substantially simultaneously, or within about minutes to hours to days to weeks and any range derivable therein, prior to and/or after administering the composition.

Administration of the composition to a cell, tissue or organism may follow general protocols for the administration of antimicrobial therapeutics, taking into account the toxicity, if any. It is expected that the treatment cycles would be repeated as necessary. In particular embodiments, it is contemplated that various additional agents may be applied in any combination with the present invention.

Pharmacological therapeutic agents and methods of administration, dosages, etc. are well known to those of skill in the art (See for example, the “Physicians Desk Reference,” Goodman & Gilman's “The Pharmacological Basis of Therapeuticsm” “Remington's Pharmaceutical Sciences,” and “The Merck Index, Eleventh Edition” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

### E. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Lipopeptide Synthesis

American Type Culture Collection (ATCC) strains as well as clinical isolates from the Canadian Intensive Care Unit (CAN-ICU) study were used, including: S. aureus ATCC 29213, methicillin-resistant Staphylococcus aureus (MRSA) ATCC 33592, S. epidermidis ATCC 14990, methicillin-resistant Staphylococcus epidermidis (MRSE) (Cefazolin-CZ MIC>32 μg/mL) CAN-ICU 61589, E. faecalis ATCC 29212, E. faecium ATCC 27270, S. pneumoniae ATCC 49619, E. coli ATCC 25922, E. coli ATCC (Gentamicin resistant-Gent R) CAN-ICU 61714, E. coli ATCC (Amikacin MIC 32 μg/mL) CAN-ICU 63074, P. aeruginosa ATCC 27853, P. aeruginosa (Gent-R) CAN-ICU 62308, S. maltophilia CAN-ICU 62584, A. baumannii CAN-ICU 63169 and K. pneumoniae ATCC 13883 (Zhanel et al., 2008).

The lipo-α-peptides and lipo-β-peptides (Tables 2 and 3) were synthesized by solid phase peptide synthesis using standard FMOC chemistry on Rink amide-4-methylbenzhydrylamine hydrochloride salt (MBHA) resin. Palmitic and Lauric acid were conjugated to the tetrapeptides via modified solid phase methods. TBTU (3 eq), lipophilic acid (3 eq) and DIEA (9 eq) reacted in a solution of 45% CH₂Cl₂ in DMF, with the process being repeated twice. Lipopeptide cleavage was achieved in 95% TFA, followed by purification on Reversed Phase C18 Silica. The homogeneity and identity of the synthetic peptides were assessed by ESI-MS, ¹H NMR and ¹³C-NMR.

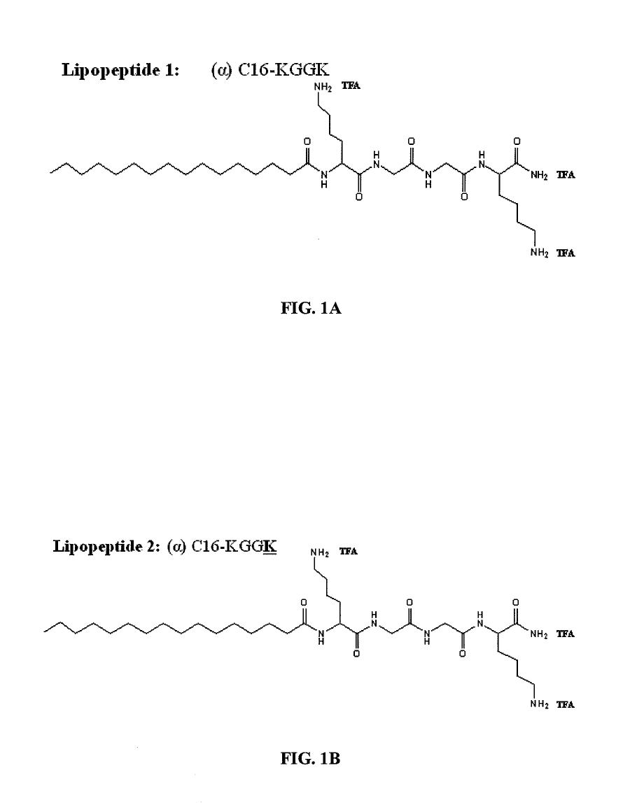

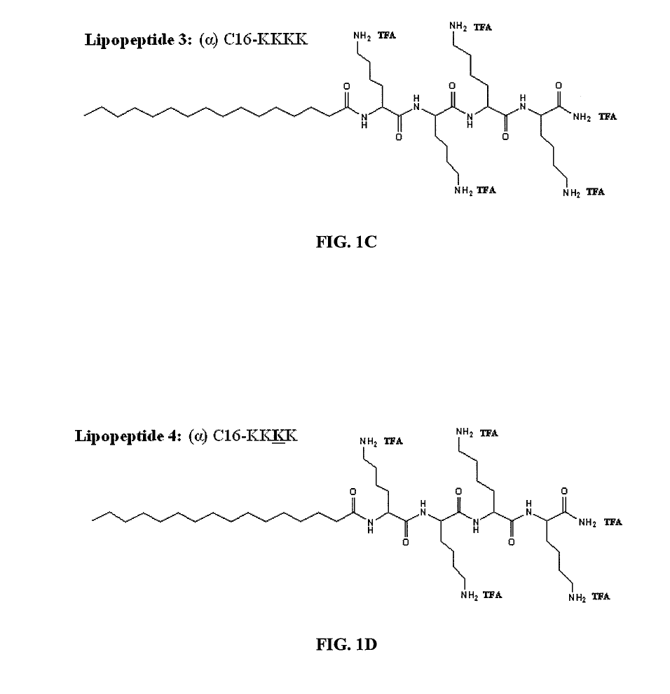

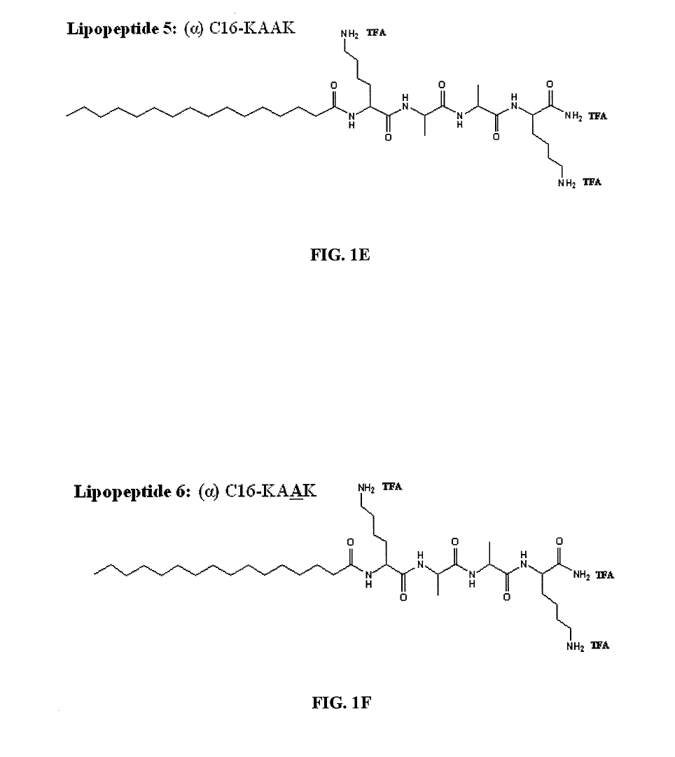

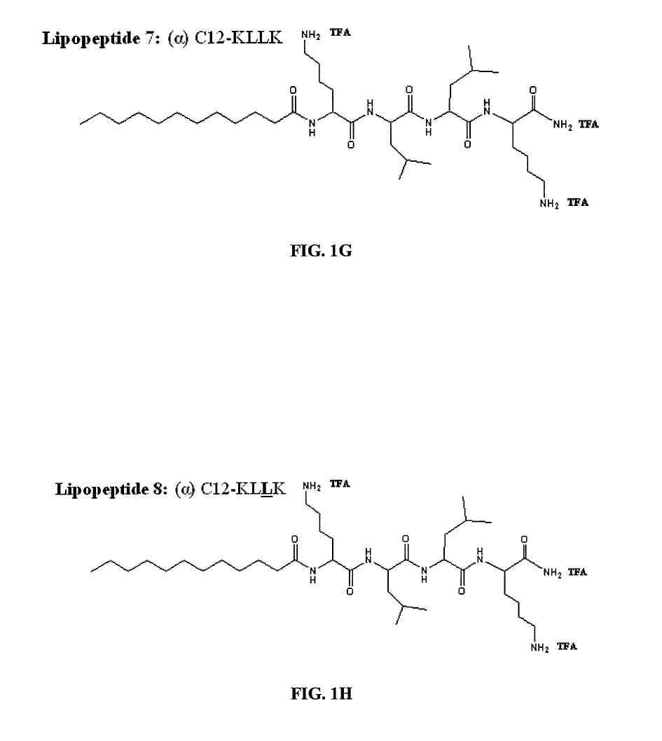

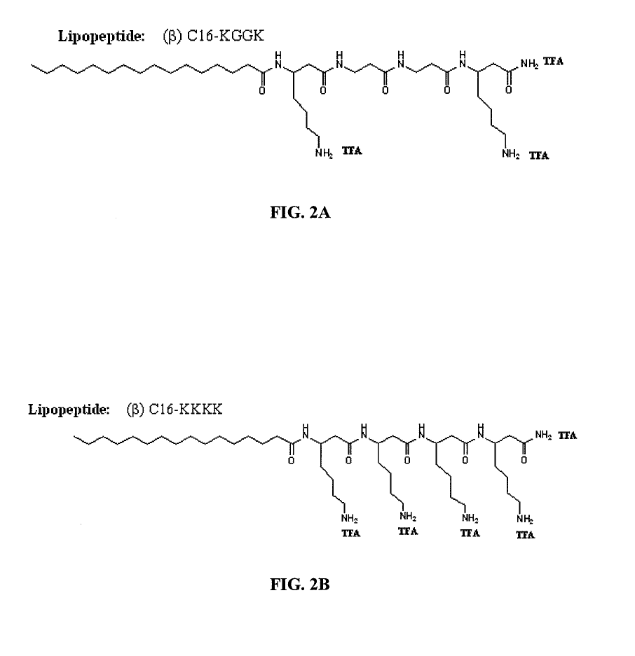

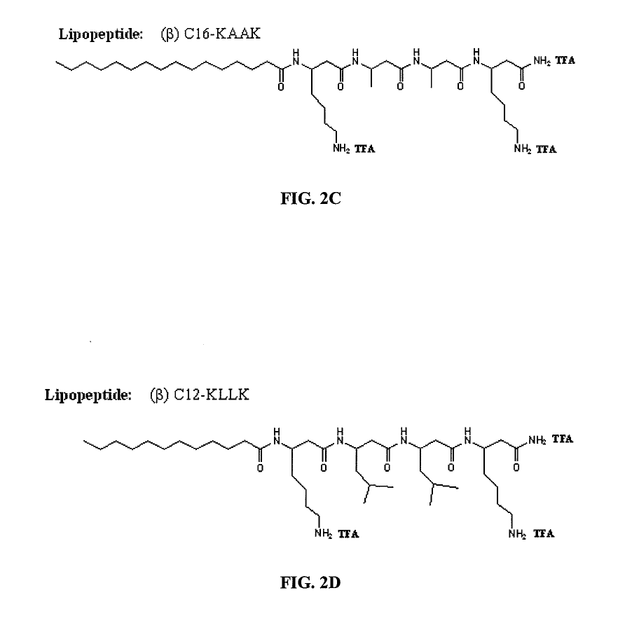

A total of 12 lipopeptides were synthesized with a tetrapeptide moiety containing (i) all L-amino acids (ii) D,L-amino acids and (iii) all β-amino acids based on the following four sequences: C16-KGGK, C16-KAAK, C16-KKKK and C12-KLLK. These sequences are based on a representative sample of the highly active N-terminal acylated lipopeptides reported by Shai and coworkers (Makovitzki et al., 2006) and as such, the D,L-amino acid based lipopeptides serve as the control group. See Tables 2 and 3. The structures of the lipo-α-peptides and the lipo-β-peptides are shown in FIGS. 1A-H and FIGS. 2A-D, respectively, where the position of the D-enantiomers is shown by underlining.

### Example 2

### Antibacterial Activity of Ultrashort Cationic Lipo-Betapeptides

Antibacterial activity against Gram positive and Gram negative microorganisms was investigated via broth macrodilution tests using CLSI methodology (Zhanel et al., 2008). Stock solutions of lipopeptide antibiotics in water were brought to a standard 512 μg/mL, with only βC12-KLLK and βC16-KAAK requiring a minute amount of DMSO. Organisms were subcultured and isolated on blood agar, suspended in 3 mL of Mueller-Hinton broth at the turbidity of a 0.5M McFarland Standard, and diluted to approximately 10⁵ CFU/mL before introduction into tubes containing serially diluted lipopeptide antibiotic in Mueller-Hinton broth. Activity testing against S. pneumoniae used broth supplemented with laked horse blood to give 5% horse blood in experimental tubes. The turbidity resulting from lipopeptide solution in broth required the creation of control tubes lacking microbes serving as turbidity controls. All tubes were incubated overnight for 16-20 h at 37° C. Colony counts of diluted 10⁵ CFU/mL solution of microorganism confirmed the validity of the trial with colony counts expected in the 10⁵ CFU/mL range and also incubated overnight in a CO₂ incubator at 37° C. and 5% CO₂.

TABLE 2
Antimicrobial Activities (MIC μg/mL) of Ultrashort Cationic α-Lipopeptides

| Control | (α)C16- | (α)C16- | (α)C16- | (α)C12- | (α)C16- | (α)C16- | (α)C16- | (α)C12- |
|---|---|---|---|---|---|---|---|---|
| Organism | KGGK | KKKK | KAAK | KLLK | KGGK | KKKK | KAAK | KLLK |
| S. aureus | 8 | 32 | 16 | 16 | 16 | 16 | 8 | 16 |
| ATCC29213 | | | | | | | | |
| MRSA | 16 | 16 | 16 | 16 | 16 | 16 | 32 | 16 |
| ATCC33592 | | | | | | | | |
| S. epidermidis | 4 | 4 | 8 | 16 | 8 | 4 | 4 | 8 |
| ATCC14990 | | | | | | | | |
| MRSE | 8 | 8 | 8 | 16 | 8 | 8 | 8 | 16 |
| CAN-ICU | | | | | | | | |
| 61589 | | | | | | | | |
| E. faecalis | 8 | 16 | 16 | 32 | 16 | 32 | 16 | 32 |
| ATCC29212 | | | | | | | | |
| E. facium | 16 | 16 | 8 | 16 | 16 | 16 | 8 | 32 |
| ATCC27270 | | | | | | | | |
| S. pneumoniae | 128 | >64 | 128 | 128 | 128 | >32 | 128 | 64 |
| ATCC49619 | | | | | | | | |
| E. coli | 16 | 16 | 16 | 128 | 16 | 32 | 32 | 64 |
| ATCC25922 | | | | | | | | |
| E. coli | 16 | 16 | 16 | 128 | 64 | 32 | 32 | 64 |
| CAN-ICU | | | | | | | | |
| 61714 | | | | | | | | |
| E. coli | 16 | 32 | 16 | 128 | 16 | 32 | 32 | 64 |
| CAN-ICU | | | | | | | | |
| 63074 | | | | | | | | |
| P. aeruginosa | 64 | 64 | 32 | 128 | 64 | 32 | 64 | 128 |
| ATCC27853 | | | | | | | | |
| P. aeruginosa | 64 | 256 | 64 | 128 | 64 | 256 | 64 | 128 |
| CAN-ICU | | | | | | | | |
| 62308 | | | | | | | | |
| S. maltophilia | 128 | 256 | 128 | >256 | 64 | 256 | 128 | >256 |
| CAN-ICU | | | | | | | | |
| 62584 | | | | | | | | |
| A. baumannii | 128 | 256 | 128 | >64 | 64 | 256 | 128 | >256 |
| CAN-ICU | | | | | | | | |
| 63169 | | | | | | | | |
| S. pneumoniae | 64 | 256 | 128 | 256 | 64 | 256 | 128 | >64 |
| ATCC13883 | | | | | | | | |

TABLE 3
Antimicrobial Activities (MIC μg/mL) of Ultrashort Cationic β-
Lipopeptides

| Control | | (β)C16- | (β)C16- | (β)C16- | (β)C12- |
|---|---|---|---|---|---|
| Organism | Gentamicin | KGGK | KKKK | KAAK | KLLK |
| S. aureus | 1 | 16 | 16 | 64 | 32 |
| ATCC29213 | | | | | |
| MRSA | 2 | 32 | 16 | 32 | 32 |
| ATCC33592 | | | | | |
| S. epidermidis | 0.25 | 8 | 4 | 8 | 16 |
| ATCC14990 | | | | | |
| MRSE | 32 | 16 | 4 | 16 | 32 |
| CAN-ICU | | | | | |
| 61589 | | | | | |
| E. faecalis | n.d. | 32 | 32 | 32 | 64 |
| ATCC29212 | | | | | |
| E. facium | n.d. | 32 | 16 | 32 | 32 |
| ATCC27270 | | | | | |
| S. pneumoniae | 4 | 128 | 128 | >64 | 128 |
| ATCC49619 | | | | | |
| E. coli | 1 | 32 | 16 | 64 | 64 |
| ATCC25922 | | | | | |
| E. coli | 128 | 32 | 32 | 64 | 64 |
| CAN-ICU | | | | | |
| 61714 | | | | | |
| E. coli | 8 | 32 | 32 | 64 | 64 |
| CAN-ICU | | | | | |
| 63074 | | | | | |
| P. aeruginosa | 8 | 64 | 64 | 256 | 128 |
| ATCC27853 | | | | | |
| P. aeruginosa | 128 | >64 | 128 | 128 | 128 |
| CAN-ICU | | | | | |
| 62308 | | | | | |
| S. maltophilia | >512 | 256 | 256 | >128 | 256 |
| CAN-ICU | | | | | |
| 62584 | | | | | |
| A. baumannii | 128 | 128 | 256 | 256 | >128 |
| CAN-ICU | | | | | |
| 63169 | | | | | |
| S. pneumoniae | 0.25 | 128 | 128 | >128 | 256 |
| ATCC13883 | | | | | |

As shown in the above Tables, the lipopeptides containing only L-amino acids did not show a significant difference in antimicrobial activity with respect to peptides incorporating the D-enantiomer of an amino acid (Papo and Shai, 2004). Also, the activities of the lipo-β-peptides were comparable to those of their D,L-amino acid counterparts, with limited differences (almost all values within 2-fold dilution; see Tables 2 and 3). Gram positive organisms proved generally more susceptible to these lipopeptide agents than did Gram negative bacteria. Among Gram negatives, only E. coli strains proved somewhat susceptible to all sequences of lipopeptides, although the MICs were higher with the C12-KLLK series, in which MIC values ranged between 64-128 m/mL. Interestingly, among Gram positives, only S. pneumoniae proved less susceptible to the lipopeptide antibiotics with MIC values largely greater than 64 μg/mL. MIC values for S. pneumoniae were reduced by 8-32 fold for all lipopeptides when the experiment was performed in Todd Hewitt instead of LHB. This indicates that lipopeptides are strongly protein-bound.

Among all species tested, S. epidermidis consistently showed the highest susceptibility to all synthesized lipopeptides, followed closely by its antibiotic resistant counterpart, MRSE. Likewise, all other organisms for which antibiotic-resistant strains were tested showed similar activities to their non-resistant counterpart. Organisms such as S. aureus, E. coli and P. aeruginosa had MIC values that for the most part, did not vary greater than a 2-fold dilution. The organisms S. maltophilia, A. baumannii and K. pneumoniae proved least susceptible to all lipopeptides.

### * * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

U.S. Pat. No. 5,593,866
U.S. Pat. No. 6,911,525
Andra et al., Biochem. J., 385:135-143, 2005.
Avrahami and Shai, Biochemistry, 42:14946-14956, 2003.
Avrahami and Shai, J. Biol. Chem. 279:12277-12285, 2004.
Coligan et al., In: Current Protocols in Immunology, Wiley Interscience, 1991, Unit 9, 1991.
Deutscher, In: Methods in Enzymology, Vol 182, Academic Press, 1990.
Goodman & Gilman's “The Pharmacological Basis of Therapeuticsm”
Greene and Wuts, In: Protective Groups in Organic Synthesis, 2^{nd} Ed.; Wiley, NY, 1999.
Japelj et al., J. Am. Chem. Soc., 129:1022-1023, 2007.
Jerala, Expert Opin. Investig. Drugs, 16:1159-1169, 2007.
Majerle et al., J. Antimicrob. Chemother., 51:1159-1165, 2003.
Makovitzki et al., Proc. Natl. Acad. Sci. USA, 103:15997-16002, 2006.
Merrifield, J. Am. Chem. Soc., 85:2149, 1963.
Papo and Shai, Biochemistry, 43:6393-6403, 2004.
Physicians Desk Reference
Remington's Pharmaceutical Sciences, 18th Ed. Mack Printing Company, 1990.
Seebach et al., Chem. Biodivers., 1:1111-1239, 2004.
Shai et al., Curr. Protein Pept. Sci., 7:479-486, 2006.
Steer et al., Curr. Med. Chem., 9:811-822, 2002.
Stewart and Young, In: Solid Phase Peptides Synthesis, Pierce, Rockford, Ill., 1984.
Straus and Hancock, Biochim. Biophys. Acta., 1758:1215-1223, 2006.
The Merck Index, Eleventh Edition
Zhanel et al., Antimicrob. Agents Chemother., 52:1430-1437, 2008.

1. A cationic Lipo-β-ultrashort peptide of formula (I): 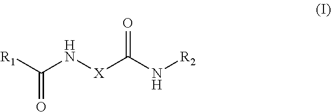
where:
R₁ is a hydrophobic group;
R₂ is H, alkyl, or an amide protecting group, and
—NH—X—C(O)— is a chain of two to four β-amino acid residues;
or a salt thereof.

2. The cationic lipo-β-ultrashort peptide of claim 1, where R₁ is alkyl_{(C5-45)} or alkenyl_{(C5-45)}.

3. The cationic lipo-β-ultrashort peptide of claim 2, where R₁ is alkyl_{(C10-18)}.

4. The cationic lipo-β-ultrashort peptide of claim 3, where R₁ is alkyl_{(C12-16)}.

5. The cationic lipo-β-ultrashort peptide of claim 1, where R₂ is H.

6. The cationic lipo-β-ultrashort peptide of claim 1, where the cationic lipo-β-ultrashort peptide is present as a trifluoroacetic acid salt.

7. The cationic lipo-β-ultrashort peptide of claim 1, where each of the two to four amino acid residues of the —NH—X—C(O)— group is selected from the group consisting of β-lysine, β-arginine, β-alanine, β-glycine, β-leucine and β-histidine.

8. The cationic lipo-β-ultrashort peptide of claim 1, where —NH—X—C(O)— is a chain of four β-amino acid residues.

9. The cationic lipo-β-ultrashort peptide of claim 8, where the chain of four (3-amino acid residues is KGGK (SEQ ID NO:1), KKKK (SEQ ID NO:2), KAAK (SEQ ID NO:3), or KLLK (SEQ ID NO:4).

10. The cationic lipo-β-ultrashort peptide of claim 1, where the chain of four β-amino acid residues is (β) C16-KGGK, (β) C16-KKKK, (β) C16-KAAK, or (β) C12-KLLK.

11. A method of making a cationic lipo-β-ultrashort peptide of formula (I): 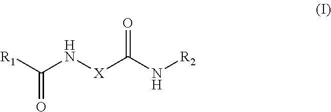
where:
R₁ is a hydrophobic group;
R₂ is H, guanidino, or an amine protecting group, and
—NH—X—C(O)— is a cationic ultrashort β-peptide;
or a salt thereof;
comprising reacting a fatty acid of formula (II): 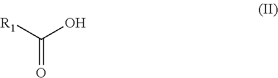
where:
R₁ is a hydrophobic group;
with the N-terminus of a cationic ultrashort β-peptide of formula (III): 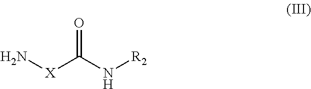
where:
R₂ is H, alkyl, or an amide protecting group, and
—NH—X—C(O)— is a cationic ultrashort β-peptide;
or a salt thereof,
under conditions to form a cationic lipo-β-ultrashort peptide of formula (I)

12. The method of claim 11, where the conditions comprise a solid-phase synthetic step.

13. The method of claim 11, where the number of equivalents of fatty acid to cationic ultrashort β-peptide ranges from about 1-5.

14. The method of claim 11, where the cationic lipo-β-ultrashort peptide is present as a trifluoroacetic acid salt.

15. A pharmaceutical composition comprising a cationic lipo-β-ultrashort peptide of formula (I): 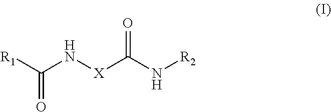
where:
R₁ is a hydrophobic group;
R₂ is H, alkyl or an amide protecting group, and
—NH—X—C(O)— is a chain of two to four β-amino acid residues;
or a salt thereof; and
a pharmaceutically acceptable carrier.

16. A method of treating a bacterial infection in a subject comprising administering to the subject an effective amount of a cationic lipo-β-ultrashort peptide of formula (I): 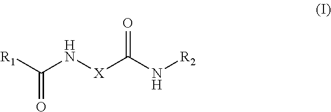
where:
R₁ is a hydrophobic group;
R₂ is H, alkyl, or an amide protecting group, and
—NH—X—C(O)— is a chain of two to four β-amino acid residues;
or a salt thereof.

17. The method of claim 16, where the bacteria causing the bacterial infection is a multi-drug resistant bacteria.

18. The method of claim 16, where the bacterial infection is caused by a Gram-positive bacteria.

19. The method of claim 18, where the Gram-positive bacteria is Staphylococcus aureus, methicillin-resistant Staphylococcus aureus (MRSA), Staphylococcus epidermidis, methicillin-resistant S. epidermidis (MRSE), Enterococcus faecalis, Enterococcus faecium, or Streptococcus pneumoniae.

20. The method of claim 19, where the Gram-positive bacteria is Staphylococcus epidermidis or methicillin-resistant Staphylococcus epidermidis (MRSE).

21. The method of claim 16, where the bacterial infection is caused by a Gram-negative bacteria.

22. The method of claim 16, where the Gram-negative bacteria is E. coli, Pseudomonas aeruginosa, Stenotrophomonas maltophilia, Acinetobacter baumannii, or Klebsiella pneumoniae

23. The method of claim 22, where the Gram-negative bacteria is E. coli.

24. The method of claim 16, where the minimum inhibitory concentration of the cationic lipo-β-ultrashort peptide (MIC) is ≦64 μg/mL.

25. The method of claim 16, further comprising administration of a second antibacterial agent.

26. The method of claim 16, further comprising diagnosing the subject as needing treatment for the bacterial infection prior to administering the cationic lipo-β-ultrashort peptide.

27. The method of claim 16, where the cationic lipo-β-ultrashort peptide is topically administered to skin of the subject, where the skin has a bacterial infection.

28. A method of preventing a bacterial infection in a subject comprising administering to the subject an effective amount of a cationic lipo-β-ultrashort peptide of formula (I): 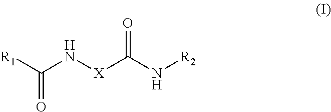
where:
R₁ is a hydrophobic group;
R₂ is H, alkyl, or an amide protecting group, and
—NH—X—C(O)— is a chain of two to four β-amino acid residues;
or a salt thereof.

29. The method of claim 28, further comprising diagnosing the subject as needing preventative treatment for the bacterial infection prior to administering the cationic lipo-β-ultrashort peptide.

30. The method of claim 28, where the cationic lipo-β-ultrashort peptide is topically administered to skin of the subject, where the skin is at risk of having a bacterial infection.

31. A method of treating a bacterial infection in a subject comprising administering to the subject an effective amount of a cationic lipo-β-ultrashort peptide of formula (I): 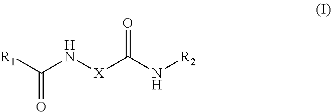
where:
R₁ is a hydrophobic group;
R₂ is H, alkyl, amide protecting group, and
—NH—X—C(O)— is a chain of two to four β-amino acid residues;
or a salt thereof.
where the effective amount of the cationic lipo-β-ultrashort peptide is less than the effective amount of the corresponding cationic lipo-α-ultrashort peptide.
